(19) United States
(12) Patent Application Publication
(10) Pub. No.: US 2008/0009064 A1
(43) Pub. Date: Jan. 10, 2008

(54) Temperature-Responsive Microcarrier

(76) Inventors: Vincent Ronfard, Newton, MA (US); Christopher S. O'Reilly, Middleboro, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/774,275

(22) Filed: Jul. 6, 2007

Related U.S. Application Data
(60) Provisional application No. 60/806,679, filed on Jul. 6, 2006.

(51) Int. Cl. C12N 5/00

(52) U.S. Cl. 435/402

(57) ABSTRACT

The invention relates to compositions and methods useful for cell culture in which cell adherence and release of cultured cells can be performed in a temperature-responsive manner.

### CROSS REFERENCE TO RELATED APPLICATIONS

This application claims the benefit of U.S. Provisional Application 60/806,679, filed on Jul. 6, 2006; the entire contents of which are hereby incorporated by this reference.

### FIELD OF THE INVENTION

The present invention relates generally to the field of cell culture, which is a laboratory process used primarily for the growth, propagation, and production of cells for analysis and the production and harvesting of cell products.

### BACKGROUND OF THE INVENTION

Living cells are usually seeded onto a plastic surface in a growth media containing many of the nutrients and growth factors present in their natural environment. The cells, sitting on the bottom of a plastic vessel, such as a Petri dish or a flask, are then placed into an incubator which provides a warm, moist, and appropriately gassed environment to grow. There is virtually no limit to the number and variety of cells that can be cultured, and valuable products and data that can be obtained from cells in culture.

The bulk of traditional cell culture depends on the use of flat bottom dishes on which cells of interest are grown. Petri dishes, and other cell culture ware, provide a surface on which anchorage dependent cells can attach and grow. A traditional Petri dish has a surface area of 78.52 cm and can support the growth of over 1×10⁶ cells when fully confluent. Improvements on the Petri dish have included the use of cell flasks, roller bottles, and growing cells on fibers in culture vessels.

Microcarriers have been developed as an alternative to growing cells on the surface of the growth media container or culture vessel. Microcarriers have been created out of a variety of materials such as plastic, glass, gelatin and calcium-alginate, in order to increase the surface area available on which cells can grow. Microcarriers have many advantages. They are essential when surfaces are needed for anchorage dependent cells. They are also inexpensive (price/m2). Microcarrier technology results in a homogeneous culture system that is truly scalable. Because of their large surface area to volume ratio, they occupy less space in storage, production and waste-handling. The surface also allows cells to secrete and deposit an extracellular matrix, which helps introduce certain growth factors to cells. Spherical microcarriers have short diffusion paths, which facilitates nutrient supply in general. The extracellular matrix also gives cells support to build their cytoskeleton and to organize organelles intracellularly, both of which may increase the yield of functional product.

A major area of application for microcarrier culture is the production of large numbers of cells. The advantages of the microcarrier system can be used to obtain high yields of cells from small culture volumes. The high yield of cells per unit culture volume and the large increase in cell number during the culture cycle (10-fold or more) makes microcarrier culture an attractive technique for producing cells from a wide range of culture volumes.

Applications for small culture volumes include situations when only a few cells are available to initiate a culture (e.g. clinical diagnosis, cloned material). Microcarriers can be used to increase the culture surface area in small volumes and at the same time keep the density of cells/mL as high as possible. Maintaining high densities of cells leads to conditioning of the culture medium and stimulation of cell growth. With traditional monolayer techniques for small cultures, it is not possible to achieve a high culture surface area/volume ratio (approx. 4 cm2/mL in Petri dishes). Microcarrier cultures provide a surface area/volume ratio of approximately 20 cm2/mL. The increase in culture surface area means that a greater yield of cells is achieved before subculturing is necessary. Microcarrier culture also provides a method for rapid scale-up with a minimum of subculture steps

Cultured cells are traditionally collected or detached from the surface of the microcarrier by treating with a proteolysis enzyme (e.g. trypsin) or a chemical material (e.g. EDTA), or both in combination. In the treatment with a proteolysis enzyme or chemical material, however, the following problems occur: (1) the treating process is complicated and there is high possibility of introducing impurities; (2) the cultured or grown cells are adversely affected by the treatment and the treatment may harm their inherent functions.

Temperature-responsive polymers are obtained by homo- or co-polymerization of monomers. Further, monomers may be copolymerized with other monomers, or one polymer may be grafted to another or two polymers may be copolymerized or a mixture of polymer and copolymer may be employed. If desired, polymers may be crosslinked to an extent that will not impair their inherent properties.

Typical examples of heat-responsive polymer materials having ester bonds or acid amide bonds include partially oxidized polyvinyl alcohol and N-isopropyl acrylamides. It is known that the cloud point of an ester bond-type polymer or an alkylamide polymer would be gradually lowered with an increase in the carbon atom number in a side chain.

In polymer compounds showing structural changes due to external stimuli (temperature, pH, light, etc.), the structural changes result in changes in the characteristics of the polymers, for example, volume or hydrophilic/hydrophobic nature. For example, it is well known that poly(N-isopropyl acrylamide) shows a structural change in an aqueous solution depending on temperature. Namely, this compound is soluble in water in a low temperature side of 32° C. or below but becomes insoluble in water in a high temperature side exceeding 32° C. That is to say, it is a temperature-responsive polymer compound having a lower critical solution temperature (LCST). It is considered that such a polymer compound would show a hydrophilic nature and be dissolved in water in a swollen state in the low temperature side and, in the high temperature side, it would show a hydrophobic nature and be aggregated in a contracted state. By using these temperature-depending changes, temperature-responsive polymer compounds have been applied to drug delivery systems and high-functional materials such as separators.

In the field of cell culture, investigators seek methods and compositions to overcome the aforementioned drawbacks and challenges as the need for large-scale cell culture capabilities increases for cell therapies, cell-products and tissue engineering applications.

### SUMMARY OF THE INVENTION

In order to overcome the above-mentioned problems, the present invention provides temperature-responsive microcarrier substrates on which cells are cultured and from which the cultured cells are collected or detached without a proteolysis enzyme or chemical material and methods for using these microcarrier substrates.

In a preferred embodiment, the invention is a cell culture substrate comprising a culture support coated with a temperature-responsive polymer wherein the support is a cell-compatible microcarrier and wherein the temperature-responsive polymer binds cells at a first temperature compatible with cell proliferation and releases cells at a second temperature that is compatible with cell viability.

In another preferred embodiment, the invention is a cell culture substrate comprising a temperature-responsive polymer wherein the polymer is in the shape of a microcarrier and wherein the temperature-responsive polymer binds cells at a first temperature compatible with cell proliferation and releases cells at a second temperature that is compatible with cell viability. In other words, the polymer itself also serves as a support rather than coated on a support material.

In an alternate preferred embodiment, the microcarrier substrate of the present invention comprises a microcarrier support and a coating thereon, wherein the coating is formed from a polymer or copolymer which has a critical solution temperature to water within the range of 0° C. to 80° C. The microcarrier preferably binds cells at a first temperature between 33-39° C. and releases cells at a second temperature less than 33° C.

In another preferred embodiment, the invention is a method for culturing cells, comprising seeding cells on a plurality of microcarrier supports coated with a temperature-responsive polymer; culturing the cells in a medium under conditions that permit binding of the cells to the microcarrier supports; releasing the cells from the microcarrier supports into the medium by changing the temperature of the microcarrier supports to a temperature that permits release of the cells from the microcarrier supports; and, if needed, separating the cells and the medium from the microcarrier supports. The cells are cultured preferably in a chemically defined medium at a temperature that permits adherence of the cells to the microcarrier supports and are released by changing the temperature to one that permits release of the cells from the microcarrier supports to obviate the need for proteolytic enzymes and/or chemical additives. The culture method of the invention may be used to culture any type of cell, particularly any type of animal cell. Cells that can be used include, but are not limited to stem cells, committed stem cells, and differentiated cells.

Examples of stem cells that can be used include but are not limited to embryonic stem cells, bone marrow stem cells and umbilical cord stem cells and stem cells derived from other tissues and organs such as from blood and skin. Other examples of cells include but are not limited to: osteoblasts, myoblasts, neuroblasts, fibroblasts, glioblasts; germ cells, hepatocytes, chondrocytes, keratinocytes, smooth muscle cells, cardiac muscle cells, connective tissue cells, epithelial cells, endothelial cells, hormone-secreting cells, cells of the immune system, and neurons. The microcarrier culture system of the present invention may also be used in the production of biological materials such as vaccines, enzymes, hormones, antibodies, interferons and nucleic acids.

### DETAILED DESCRIPTION OF THE INVENTION

Microcarrier culture is a versatile technique for growing animal cells and can be used in a variety of ways for a wide range of applications. Although microcarrier culture is an advanced technique, it is based on standard animal cell culture procedures and does not require complicated or sophisticated methods. The possible uses of the microcarrier technology of this invention fall into three categories: a) high-yield production of cells, viruses or cell products, b) in vitro cell studies, and c) routine cell culture techniques. As used herein, the terms “microcarriers”, “cell-culture microcarriers” and “cell-growth microcarriers” mean small, discrete particles suitable for cell attachment and growth.

The critical solution temperature is defined as follows. When a certain material is mixed with water, the mixture is divided into two layers at a particular temperature because of its poor solubility, but eventually the material is completely dissolved with water to turn it to a uniform solution if it is either heated or cooled beyond a certain temperature. The certain temperature is defined as “critical solution temperature”. If the uniform solution is formed when heated, the critical solution temperature is called “upper critical solution temperature”. If the uniform solution is formed when cooled, it is called the “lower critical solution temperature”.

The critical solution temperature is obtained by making a solution phase diagram with respect to water (ion exchanged water or distilled water). For making the solution phase diagram, mixtures of a polymer to be measured and water in various concentrations (such as weight %, volume %, molar %, molar ratio, etc.) are prepared and the mixtures are heated or cooled to observe the conditions of the mixture. The conditions are determined by art-known methods, such as (a) visual observation, (b) critical opalescence, (c) scattered light strength, and (d) transmitted laser light measure and the like.

The temperature-responsive polymer or copolymer of the present invention should have either an upper or lower critical solution temperature within the range of 0° C. to 80° C., preferably 20° to 50° C. If the temperature is too high, cultured or grown cells may die; if too low, the growth rate of the cells may be very much lowered or the cells may die. In one embodiment of the invention, the temperature-responsive polymer selected is one that binds or causes cells, such as human dermal fibroblasts, to adhere at temperatures corresponding with temperatures optimal to growth of human derived cells, such as between 33° C. to 39° C. and causes such cells to detach at temperatures between 0° C. and 33° C. For example, an N-substituted (meth)acrylamide such as N-isopropyl acrylamide with a lower critical solution homopolymer temperature of 32° C. may be selected.

The polymer or copolymer of the present invention may be prepared by polymerizing or copolymerizing hydrophilic monomers. Non-limiting examples of the monomers, provided that a parenthesis indicates a lower critical solution temperature of homopolymer, are represented by a (meth)acrylamide, such as acrylamide, methacrylamide, etc.; an N-substituted (meth)acrylamide, such as N-ethyl acrylamide (72° C.), N-n-propyl acrylamide (21° C.), N-n-propyl methacrylamide (27° C.), N-isopropyl acrylamide (32° C.), N-isopropyl methacrylamide (43° C.), N-cyclopropyl acrylamide (45° C.), N-cyclopropyl methacrylamide (60° C.), N-ethoxyethyl acrylamide (about 35° C.), N-ethoxyethyl methacrylamide (about 45° C.), N-tetrahydrofurfuryl acrylamide (about 28° C.), N-tetrahydrofurfuryl methacrylamide (about 35° C.) etc.; N,N-di-substituted (meth)acrylamide, such as N,N-dimethyl(meth)acrylamide, N,N-ethylmethyl acrylamide (56° C.), N,N-diethyl acrylamide (32° C.), 1-(1-oxo-2-propenyl)-pyrrolidine (56° C.), 1-(1-oxo-2-propenyl)-piperidine (about 6° C.), 4-(1-oxo-2-propenyl)-morpholine, 1-(1-oxo-2-methyl-2-propenyl)-pyrrolidine, 1-(1-oxo-2-methyl-2-propenyl)-piperidine, 4-(1-oxo-2-methyl-2-propenyl)-morpholine etc.; a vinyl ether, such as methyl vinyl ether (35° C.); and the like. A copolymer of the above listed monomers or other monomers, a graft polymer or copolymer or a mixture of the polymers can also be employed in the present invention, in order to adjust the critical solution temperature, depending upon the type of cells, to enhance an interaction between the support and the coating thereon or to control the balance between the hydrophilic and hydrophobic properties of the bed material. The polymer or copolymer of the present invention may be crosslinked unless the inherent properties of the polymer would be deleteriously affected thereby.

A microcarrier support may be required depending on the size, shape or other physical property of the microcarrier or temperature-responsive polymer. The microcarrier support of the present invention can be prepared from any material, for example polymers (e.g. polystyrene, poly(methyl methacrylate, polyethylene, polyester, polypropylene, polycarbonate, polyvinyl chloride, polyvinylidene, polydimethylsiloxene, fluoropolymers, fluorinated ethylene propylene, etc.), temperature-responsive polymers (e.g. poly(N-isopropyl acrylamide), poly(N-isopropyl methacrylamide), poly(N-n-propyl acrylamide) or poly(N,N-diethyl acrylamide), etc.) ceramics, metals including stainless steel, glass and modified glass, silicone substrates including silica, fused silica, polysilicon or silicon crystals, silicone rubber, cellulose, dextran, collagen (gelatin), and glycosaminoglycans as well as substances that can generally be given shape, for example, polymer compounds other than those listed above. Alternatively, the microcarriers of the present invention are made from a temperature-responsive polymer in the shape of a microcarrier without a support. In other words, the temperature-responsive polymer is self-supporting and is generally a spherical droplet of the polymer without a supportive core made of a material different from the temperature-responsive polymer. The temperature-responsive polymer in this self-supporting configuration may be combined or admixed with other substances to improve cohesion of the polymer molecules and this improve the structural integrity of the microcarrier. These materials may be formed into different microcarrier shapes. Spherical is the most preferred shape, but fibers, flat discs, woven discs, cubes and other shapes may also be used. Textures may also be formed on the surface of the microcarrier to provide control of surface area and cell interaction. Grooves, channels, pits formed on the microcarrier surfaces provide increased surface area over smooth surfaces.

The diameter of the different microcarriers varies from 10 μm up to 5 mm. The smaller are best suited for stirred tanks, whereas the higher sedimentation rates of the larger make them suitable for fluidized and packed beds. The smaller the microcarriers, the larger the surface in the settled bed volume because of the smaller void volume between them. The ideal size for smooth microcarriers is 100-300 μm. A very narrow size distribution is most important for good mixing in the reactor and an equal sedimentation of the beads during scale-up steps in large-scale processes.

A polymer or copolymer can be bound on the support by a chemical method or by a physical method. In the chemical method an electron beam, gamma ray irradiation, ultraviolet irradiation, corona treatment and plasma treatment can be used. In case where the support and the coating have groups reactive with each other, an organic reaction (e.g. a radical, anionic or cationic reaction) can also be used. In the physical method, the polymer per se or a combination of the polymer and a matrix compatible with the support is coated on the support, thus binding by physical absorption power. Examples of the matrix are graft or block copolymers of the polymer to be coated, with the monomer forming the support or other monomers compatible with the support.

In order to collect or detach the grown or cultured cells, the microcarrier substrates are either heated or cooled to exceed the upper or lower critical solution temperature, thus detaching the cells, and the microcarriers are rinsed with an isotonic solution to collect the cells. The means for changing the temperature of the microcarrier substrates depends on the vessel in which the cells are cultured. If the vessel is usually cultured in an incubator, the vessel may simply be removed from the incubator to the outside room at room temperature or placed in a refrigerator. If the vessel is a stirred-cell bioreactor with environmental controls, the internal settings may be reset to change the temperature conditions of the culture, for example, by changing the temperature of the culture medium. Alternatively, the warm culture medium may be removed and replaced with cooled medium at a temperature sufficient to cause release of the cells from the microcarrier material. Other means for changing the temperature of a culture system will depend on the size, volume and construction of the vessel and would be easily ascertained by one of skill in the art of cell culture without undue experimentation.

The cell culture method of the invention comprises seeding cells on a plurality of sterile microcarrier supports coated with a temperature-responsive polymer or sterile temperature-responsive microcarrier supports; culturing the cells in a medium under conditions that permit binding of the cells to the microcarrier supports; releasing the cells from the microcarrier supports into the medium by changing the temperature of the microcarrier supports to a temperature that permits release of the cells from the microcarrier supports; and separating the cells and the medium from the microcarrier supports. This method will be illustrated using poly(N-isopropyl acrylamide) as a coating on a microcarrier support for the culture of human dermal fibroblasts in defined medium as an illustration of one embodiment of the invention. Poly(N-isopropyl acrylamide) has a lower critical solution temperature of about 32° C. in water. The monomer, i.e. N-isopropyl acrylamide, is polymerized on polystyrene microcarrier beads for cell culture by irradiating electron beams. At temperatures higher than 32° C., the poly(N-isopropyl acrylamide) coating is hydrophobic and expels water molecules inside the coatings, which results in a reduced volume. At temperatures lower than 32° C., the coating is hydrophilic and holds water molecules to result in swelling.

The culture method of the invention is one that employs chemically defined medium and avoids the use of proteolytic enzymes and/or chemicals in the culture and release of cells from the microcarrier substrates. Culture media formulations suitable for use in the present invention are selected based on the cell types to be cultured and the tissue structure to be produced. The culture medium that is used and the specific culturing conditions needed to promote cell growth, cell-product synthesis, and viability will depend on the type of cell being grown.

The use of chemically defined culture media is preferred, that is, media free of undefined animal organ or tissue extracts, for example, serum, pituitary extract, hypothalamic extract, placental extract, or embryonic extract or proteins and factors secreted by feeder cells. In a more preferred embodiment, the media is free of undefined components and defined biological components derived from non-human animal sources. In a most preferred embodiment, human and animal derived compounds are replaced with their recombinant-derived structural and/or functional equivalents. When the invention is carried out utilizing screened human cells cultured using chemically defined components derived from no non-human animal sources or recombinant-derived equivalents, the resultant cell culture is a defined human cell culture. Synthetic functional equivalents may also be added to supplement chemically defined media within the purview of the definition of chemically defined for use in the most preferred fabrication method. Generally, one of skill in the art of cell culture will be able to determine suitable natural human, human recombinant, or synthetic equivalents to commonly known animal components to supplement the culture media of the invention without undue investigation or experimentation. The advantages in using such a cell culture or its products in the clinic is that the concern of adventitious animal or cross-species virus contamination and infection is diminished. In a testing scenario, the advantages of a chemically defined construct is that when tested, there is no chance of the results being confounded due to the presence of the undefined components.

Culture medium is comprised of a nutrient base usually further supplemented with other components. The skilled artisan can determine appropriate nutrient bases in the art of animal cell culture with reasonable expectations for successfully producing a tissue construct of the invention. Many commercially available nutrient sources are useful on the practice of the present invention. These include commercially available nutrient sources which supply inorganic salts, an energy source, amino acids, and B-vitamins such as Dulbecco's Modified Eagle's Medium (DMEM); Minimal Essential Medium (MEM); M199; RPMI 1640; Iscove's Modified Dulbecco's Medium (EDMEM). Minimal Essential Medium (MEM) and M199 require additional supplementation with phospholipid precursors and non-essential amino acids. Commercially available vitamin-rich mixtures that supply additional amino acids, nucleic acids, enzyme cofactors, phospholipid precursors, and inorganic salts include Ham's F-12, Ham's F-10, NCTC 109, and NCTC 135. Albeit in varying concentrations, all basal media provide a basic nutrient source for cells in the form of glucose, amino acids, vitamins, and inorganic ions, together with other basic media components. The most preferred base medium of the invention comprises a nutrient base of either calcium-free or low calcium Dulbecco's Modified Eagle's Medium (DMEM), or, alternatively, DMEM and Ham's F-12 between a 3-to-1 ratio to a 1-to-3 ratio, respectively.

The base medium is supplemented with components such as amino acids, growth factors, and hormones. Defined culture media for the culture of cells of the invention are described in U.S. Pat. No. 5,712,163 to Parenteau and in International PCT Publication No. WO 95/31473, the disclosures of which are incorporated herein by reference. Other media are known in the art such as those disclosed in Ham and McKeehan, Methods in Enzymology, 58:44-93 (1979), or for other appropriate chemically defined media, in Bottenstein et al., Methods in Enzymology, 58:94-109 (1979). In the preferred embodiment, the base medium is supplemented with the following components known to the skilled artisan in animal cell culture: insulin, transferrin, triiodothyronine (T3), and either or both ethanolamine and o-phosphoryl-ethanolamine, wherein concentrations and substitutions for the supplements may be determined by the skilled artisan.

Insulin is a polypeptide hormone that promotes the uptake of glucose and amino acids to provide long term benefits over multiple passages. Supplementation of insulin or insulin-like growth factor (IGF) is necessary for long term culture as there will be eventual depletion of the cells' ability to uptake glucose and amino acids and possible degradation of the cell phenotype. Insulin may be derived from either animal, for example bovine, human sources, or by recombinant means as human recombinant insulin. Therefore, a human insulin would qualify as a chemically defined component not derived from a non-human biological source. Insulin supplementation is advisable for serial cultivation and is provided to the media at a wide range of concentrations. A preferred concentration range is between about 0.1 μg/ml to about 500 μg/ml, more preferably at about 5 μg/ml to about 400 μg/ml, and most preferably at about 375 μg/ml. Appropriate concentrations for the supplementation of insulin-like growth factor, such as IGF-1 or IGF-2, may be easily determined by one of skill in the art for the cell types chosen for culture.

Transferrin is in the medium for iron transport regulation. Iron is an essential trace element found in serum. As iron can be toxic to cells in its free form, in serum it is supplied to cells bound to transferrin at a concentration range of preferably between about 0.05 to about 50 μg/ml, more preferably at about 5 μg/ml. Recombinant transferring, such as human recombinant transferrin may be substituted for transferrin purified from blood sources.

Triiodothyronine (T3) is a basic component and is the active form of thyroid hormone that is included in the medium to maintain rates of cell metabolism. Triiodothyronine is supplemented to the medium at a concentration range between about 0 to about 400 ρM, more preferably between about 2 to about 200 ρM and most preferably at about 20 ρM.

Either or both ethanolamine and o-phosphoryl-ethanolamine, which are phospholipids, are added whose function is an important precursor in the inositol pathway and fatty acid metabolism. Supplementation of lipids that are normally found in serum is necessary in a serum-free medium. Ethanolamine and o-phosphoryl-ethanolamine are provided to media at a concentration range between about 10⁻⁶ to about 10⁻² M, more preferably at about 1×10⁻⁴ M.

Throughout the culture duration, the base medium is additionally supplemented with other components to induce synthesis or differentiation or to improve cell growth such as hydrocortisone, selenium, and L-glutamine.

Hydrocortisone has been shown in keratinocyte culture to promote keratinocyte phenotype and therefore enhance differentiated characteristics such as involucrin and keratinocyte transglutaminase content (Rubin et al., J. Cell Physiol., 138:208-214 (1986)). Therefore, hydrocortisone is a desirable additive in instances where these characteristics are beneficial such as in the formation of keratinocyte sheet grafts or skin constructs. Hydrocortisone may be provided at a concentration range of about 0.01 μg/ml to about 4.0 μg/ml, most preferably between about 0.4 μg/ml to 16 μg/ml.

Selenium is added to serum-free media to resupplement the trace elements of selenium normally provided by serum. Selenium may be provided at a concentration range of about 10⁻⁹ M to about 10⁻⁷ M; most preferably at about 5.3×10⁻⁸ M.

The amino acid L-glutamine is present in some nutrient bases and may be added in cases where there is none or insufficient amounts present. L-glutamine may also be provided in stable form such as that sold under the mark, GlutaMAX-1™ (Gibco BRL, Grand Island, N.Y.). GlutaMAX-1™ is the stable dipeptide form of L-alanyl-L-glutamine and may be used interchangeably with L-glutamine and is provided in equimolar concentrations as a substitute to L-glutamine. The dipeptide provides stability to L-glutamine from degradation over time in storage and during incubation that can lead to uncertainty in the effective concentration of L-glutamine in medium. Typically, the base medium is supplemented with preferably between about 1 mM to about 6 mM, more preferably between about 2 mM to about 5 mM, and most preferably 4 mM L-glutamine or GlutaMAX-1™.

Growth factors such as epidermal growth factor (EGF) may also be added to the medium to aid in the establishment of the cultures through cell scale-up and seeding. EGF in native form or recombinant form may be used. Human forms, native or recombinant, of EGF are preferred for use in the medium when fabricating a skin equivalent containing no non-human biological components. EGF is an optional component and may be provided at a concentration between about 1 to 15 ng/mL, more preferably between about 5 to 10 ng/mL.

The medium described above is typically prepared as set forth below. However, it should be understood that the components of the present invention may be prepared and assembled using conventional methodology compatible with their physical properties. It is well known in the art to substitute certain components with an appropriate analogous or functionally equivalent acting agent for the purposes of availability or economy and arrive at a similar result. Naturally occurring growth factors may be substituted with recombinant or synthetic growth factors that have similar qualities and results when used in the performance of the invention.

Media in accordance with the present invention are sterile. Sterile components are bought sterile or rendered sterile by conventional procedures, such as filtration, after preparation. Proper aseptic procedures were used throughout the following Examples. DMEM and Ham's F-12 are first combined and the individual components are then added to complete the medium. Stock solutions of all components can be stored at −20° C., with the exception of nutrient source that can be stored at 4° C. All stock solutions are prepared at 500× final concentrations listed above. A stock solution of insulin, transferrin and triiodothyronine is prepared as follows: triiodothyronine is initially dissolved in absolute ethanol in 1N hydrochloric acid (HCl) at a 2:1 ratio. Insulin is dissolved in dilute HCl (approximately 0.1N) and transferrin is dissolved in water. The three are then mixed and diluted in water to a 500× concentration.

Ethanolamine and o-phosphoryl-ethanolamine are dissolved in water to 500× concentration and are filter sterilized. Progesterone is dissolved in absolute ethanol and diluted with water. Hydrocortisone is dissolved in absolute ethanol and diluted in phosphate buffered saline (PBS). Selenium is dissolved in water to 500× concentration and filter sterilized. EGF is purchased sterile and is dissolved in PBS. Adenine is difficult to dissolve but may be dissolved by any number of methods known to those skilled in the art. Serum albumin may be added to certain components in order to stabilize them in solution and are presently derived from either human or animal sources.

For example, recombinant human albumin, human serum albumin (HSA) or bovine serum albumin (BSA) may be added for prolonged storage to maintain the activity of the progesterone and EGF stock solutions. The medium can be either used immediately after preparation or, stored at 4° C. If stored, EGF should not be added until the time of use. While not wishing to be bound by theory, supplementing the medium with amino acids involved in protein synthesis conserves cellular energy by not requiring the cells produce the amino acids themselves. Substitute and supplemental agents that are cell-compatible, defined to a high degree of purity and are free of contaminants may also be added by the skilled artisan to the medium for the qualities that they may impart to the culture performance. These agents may include polypeptide growth factors, transcription factors or inorganic salts. Cultures are maintained in a vessel to ensure sufficient environmental conditions of controlled temperature, humidity, and gas mixture for the culture of cells.

Preferred conditions for many normal human cells are between about 34° C. to about 38° C., more preferably 37±1° C. with an atmosphere between about 5-10±1% CO₂ and a relative humidity (Rh) between about 80-90%. For the purpose of illustration, normal human fibroblasts are cultured in the medium described in a stirred spinner flask according to the method described below.

Prepared autoclaved spinner flasks and side arm caps are assembled in the sterile field of a biological safety cabinet at room temperature (about 20° C). Sterile temperature responsive microcarrier beads fabricated from polystyrene and coated with poly(N-isopropyl acrylamide) are added to the flask and are allowed to settle out in a volume of chemically defined base medium (about 100-200 mL/gm of beads) and as much of the medium as possible is aspirated off without losing microcarrier beads. The vessel is rinsed twice with fully supplemented chemically defined medium prepared as described above. The volume of supplemented medium desired for first day of culture is added using ½ of final working volume for culture.

The prepared spinner flask(s) are placed into a 37±1.0° C. with 95% air/5%±1.0% CO2 incubator on a spinner base set to 20 rpm. The side arm caps are loosened and the media is allowed to equilibrate with the internal gas concentrations and incubator temperature for at least 2-24 hours before addition of cells. When equilibrated with the incubator temperature, the temperature-responsive microcarrier beads will be at about 37±1.0° C. and will bind with cells when added. Back in the sterile field, fibroblast cells are added to the vessel to seed the beads and the vessel is returned to the incubator to culture the cells. Medium exchanges are made every 2-3 days with fresh medium that has been warmed to the incubator temperature. Cells are cultured until confluent for either harvest or to expand their numbers more by adding more beads to the vessel and increasing the medium volume in the vessel. To harvest the cells, the temperature of the system is decreased to release the cells and the cells and medium are separated from the beads. To expand the cell numbers, the temperature of the system is decreased to release the cells and additional beads and a larger volume of culture medium is added to the vessel and the vessel is returned to the incubator for further culturing.

In alternate embodiment of the present invention a Wave Bioreactor® system is used to culture cells. Wave Bioreactors® produce a very low shear environment while maintaining excellent mixing and oxygenation, and are ideal for the use of microcarriers. The volume in a Wave Bioreactor® can be increased by a factor of 10, therefore a microcarrier culture can be started at a very small volume with high density of both microcarriers and cells for better cell to microcarrier contact. Media can then be added to bring the culture to final volume. After the cells have been cultured for a sufficient amount of time, they are released from the culture substrate by lowering the temperature of the culture environment.

Cell release and adherence are temperature controlled. According to the present invention, the surface of the microcarrier material reversibly changes from hydrophilic to hydrophobic, and vice versa, by controlling the temperature. Accordingly, the grown or cultured cells are detached from the microcarrier material by simply controlling the temperature without destroying the cells, and then rinsed with an isotonic solution to collect the cells.

Alternatively, the warm culture medium may be removed and replaced with cooled medium at a temperature sufficient to cause release of the cells from the microcarrier material. Since the method of the present invention does not employ a proteolysis enzyme (such as trypsin) and a chemical material (such as EDTA), the detachment or removal process is simplified and virtually no impurities are introduced. Furthermore, the method of the present invention does not enzymatically or chemically injure the cells thus protecting the integrity and inherent functions of the cells. The release method of the present invention may be used to separate cells prior to sub-culturing or scaling-up. Cells harvested from one microcarrier culture can be used directly to inoculate the next culture containing fresh microcarriers. For one sub-culture cycle, it is possible when scaling up to release cells from the microcarriers using the temperature release method of the present invention and then to add fresh microcarriers. In this way, the culture contains old and new microcarriers.

To separate the released cells in medium from the microcarrier substrates, a mesh or filter may be employed. Alternatively, centrifugation methods may be employed. If the microcarrier substrates are neutrally buoyant or buoyant, the microcarrier substrates will float and the cells will sink so that they may be collected by aspiration from the bottom of the vessel or by skimming the microcarrier substrates from the surface of the medium. Once the microcarrier substrates are separated from the cells in medium, the cells are concentrated using centrifugation. Once concentrated, the medium is removed to the extent possible and the cells are resuspended in fresh medium and passaged to new culture vessels or incorporated into a cell-therapy for treating a subject in need of cell therapy.

Filtration of the cultured cells from the beads to collect the cells is generally desirable in the culture method. Filtration means include, but are not limited to: standard macro-filtration, techniques and apparatuses, such as flat bed vacuum filtration, dead end filtration, and by other techniques known in the art of filtration. In one filtration method, tangential flow filtration is a preferred means of filtration. Tangential flow filtration requires continuous pumping of the cells and beads within a volume of medium through the bore of a filter in the filtration loop to avoid caking and clogging the filter by the larger beads. Because the cells in this invention are generally shear sensitive and would easily become damaged in pumping equipment and standard valves, the method includes an effective filtration technique to filter the beads and from the cells.

An example of a tangential flow filtration system of the present invention is a closed loop system comprising a feed tank, an outlet of which is connected in series with a peristaltic feed pump, which in turn is connected in series to a filtration module, which in turn is connected in series to a valve, which is connected in series to the inlet of the feed tank. The feed tank allows for a continuous feed to maintain system volume as filtrate is removed. Pressure valves are connected in-line located on either side of the filtration module. The filtration module comprises an inlet and an outlet in line with the filtration loop. On the opposite side of the filter, a second outlet removes filtrate from the closed loop system.

Culture medium containing cells and temperature responsive microcarriers are pumped from the culture containers into the feed tank of the filtration system. The cells may be released from the microcarriers prior to pumping into the feed tank using any of the release methods described, or the filtration system may be cooled to a temperature sufficient to cause release of the cells from the microcarrier material prior to and/or during filtration. Once the feed tank had a sufficient volume of microcarrier culture media the pump is turned on. When the media is circulating, the media is pumped tangentially along the surface of the membrane. An applied pressure serves to force a portion of the media and cells through the membrane to the filtrate side while microcarriers, which are too large to pass through the membrane pores are retained on the upstream side, swept along by the tangential flow, and thus remained in circulation without build up at the surface of the membrane. The pump is left on to conduct circulation of media through the filtration circuit. During each pass of media over the surface of the membrane, the applied pressure forces a portion of the cell media through the membrane and into the filtrate stream.

In another separation method of the present invention, the microcarriers are manufactured using materials that allow magnetic separation of released cells and microcarriers. For example, a ferro-magnetic core may be provided in each microcarrier allowing an applied magnetic field to be used to separate the microcarriers from the cells and media. In another example, paramagnetic microcarriers (which only exhibit magnetic properties when placed within a magnetic field) are used. In the first, tube-based method, paramagnetic microcarriers are removed from the cell suspension using an external magnet that draws the microcarriers to the inner edge of the tube, allowing the cells and media to be removed. Removing the tube from the magnetic field releases the microcarriers. Separation is gentle and does not require centrifugation or columns. In the alternative, column-based method, the cells, media and paramagnetic microcarriers pass through a separation column, which is placed in a strong, permanent magnet. The column matrix serves to create a high-gradient magnetic field that retains the paramagnetic microcarriers while cells and media flow through.

In another separation method cells are recovered by fluidized bed separation. A fluidized bed system can be used to separate cells from microcarriers or microcarrier with cells. A possible reactor design is a conical shape. The fluid stream comes from the bottom and harvest occurs from the top. A top filter with mesh size reduces the risk of losing microcarriers from the system.

In still another separation method cells are recovered using a vibromixer. A vibromixer consists of a disk with conical perforations, perpendicularly attached to a shaft moving up and down with a controlled frequency and amplitude. Increasing the amplitude and the frequency of the translatory movement, increases the turbulence in the reactor. Vibromixers do not require a dynamic sealing, are a closed system and are therefore considered as very safe regarding containment. Mixing is efficient and gentle (low shear force).

At larger scale, a special harvesting reactor may be used. The vessel is divided into two compartments by a stainless steel mesh filter. The upper compartment contains a vibromixer, a reciprocating plate with holes moving at a frequency of 50 Hz. The microcarriers are collected on top of the mesh. Cell release is achieved by adding cooled washing buffer and draining it through the mesh. Additional cooled washing buffer may be added so that it just covers the microcarriers and left for some minutes (depending on the cell line). The Vibromixer may then be used for a short time to help mix the buffer solution and microcarriers. After release, the cells separate from the used microcarriers by draining through the mesh. It is also possible to only detach the cells and to transfer the entire mixture of used beads and cells to the new reactor.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims. All cited references are, hereby, incorporated by reference.

1. A method for culturing cells, comprising:
seeding cells on a plurality of cell-compatible microcarrier supports coated with a temperature-responsive polymer or copolymer;
culturing the cells in a medium under conditions that permit binding of the cells to the cell-compatible microcarrier supports;
releasing the cells from the cell-compatible microcarrier supports into the medium by changing the temperature of the cell-compatible microcarrier supports to a temperature that permits release of the cells from the cell-compatible microcarrier supports; and
separating the cells from the cell-compatible microcarrier supports.

2. The method of claim 1, wherein the medium is chemically defined.

3. The method of claim 1, wherein the method uses no enzymes or chemical additives to release the cells.

4. The method of claim 1, wherein the cells are osteoblasts, myoblasts, neuroblasts, fibroblasts, glioblasts; germ cells, hepatocytes, chondrocytes, keratinocytes, smooth muscle cells, cardiac muscle cells, connective tissue cells, epithelial cells or endothelial cells.

5. The method of claim 1, wherein the temperature-responsive polymer or copolymer is selected from the group consisting of poly(N-isopropyl acrylamide), poly(N-isopropyl methacrylamide), poly(N-n-propyl acrylamide) or poly(N,N-diethyl acrylamide).

6. The method of claim 1, wherein the cell-compatible microcarrier support material is selected from the group consisting of polymers, ceramics, metals, glass, silicone substrates, silicone rubber, cellulose, dextran, collagen (gelatin), and glycosaminoglycans.

7. A cell culture substrate comprising:
a support coated with a temperature-responsive polymer or copolymer;
wherein the support is a cell-compatible microcarrier;
wherein the temperature-responsive polymer binds cells at a first temperature compatible with cell proliferation and releases cells at a second temperature that is compatible with cell viability.

8. The cell culture substrate of claim 7, wherein the first temperature is between 33-39° C. and the second temperature is less than 33° C.

9. The cell culture substrate of claim 7, wherein the first temperature is between 33-39° C. and the second temperature is greater than 39° C.

10. The cell culture substrate of claim 7, wherein the first temperature is compatible with cell proliferation and the second temperature is between 20° to 50° C.

11. The cell culture substrate of claim 7, wherein the cell-compatible microcarrier is spherical, elongate, tubular, disk/wafer, cuboidal, or contains pores.

12. The cell culture substrate of claim 7, wherein the temperature-responsive polymer or copolymer is selected from the group consisting of poly(N-isopropyl acrylamide), poly(N-isopropyl methacrylamide), poly(N-n-propyl acrylamide) or poly(N,N-diethyl acrylamide).

13-15. (canceled)

16. The cell culture substrate of claim 19, wherein the cells are osteoblasts, myoblasts, neuroblasts, fibroblasts, glioblasts; germ cells, hepatocytes, chondrocytes, keratinocytes, smooth muscle cells, cardiac muscle cells, connective tissue cells, epithelial cells or endothelial cells.

17. (canceled)

18. The cell culture substrate of claim 19, wherein the cell culture substrate additionally comprises additional temperature-responsive cell-compatible microcarrier supports.

19. A cell culture substrate comprising:
a temperature-responsive polymer or copolymer support;
wherein the support is a cell-compatible microcarrier;
wherein the temperature-responsive polymer binds cells at a first temperature compatible with cell proliferation and releases cells at a second temperature that is compatible with cell viability.

20. The cell culture substrate of claim 19, wherein the first temperature is between 33-39° C. and the second temperature is less than 33° C.

21. The cell culture substrate of claim 19, wherein the first temperature is between 33-39° C. and the second temperature is greater than 39° C.

22. The cell culture substrate of claim 19, wherein the first temperature is compatible with cell proliferation and the second temperature is between 20° to 50° C.

23. The cell culture substrate of claim 19, wherein the cell-compatible microcarrier is spherical, elongate, tubular, disk/wafer, cuboidal, or contains pores.

24. The cell culture substrate of claim 19, wherein the temperature-responsive polymer or copolymer is selected from the group consisting of poly(N-isopropyl acrylamide), poly(N-isopropyl methacrylamide), poly(N-n-propyl acrylamide) or poly(N,N-diethyl acrylamide).

25. (canceled)
